(19) United States
(12) Patent Application Publication
(10) Pub. No.: US 2011/0009761 A1
(43) Pub. Date: Jan. 13, 2011

(54) Method and system for real-time profiling respiratory motion with variable-horizon prediction capability

(76) Inventors: Dan Ruan, Palo Alto, CA (US); Paul J. Keall, Palo Alto, CA (US)

Correspondence Address: LUMEN PATENT FIRM, 350 Cambridge Avenue, Suite 100, PALO ALTO, CA, 94306, US

(21) Appl. No.: 12/803,967

(22) Filed: Jul. 9, 2010

Related U.S. Application Data
(60) Provisional application No. 61/270,648, filed on Jul. 9, 2009.

(51) Int. Cl. A61B 5/08

(52) U.S. Cl. 600/529

(57) ABSTRACT

A method of profiling respiratory motion is provided that includes estimating a temporal respiratory pattern, using a warping function to map the temporal respiratory pattern to a corresponding phase value, using a baseline drift function to determine drift in the temporal respiratory pattern, and noise filtering the temporal respiratory pattern, where variations in a respiratory motion are provided. The warping function includes combining an elliptical shape prior for providing iso-phase events in real-time, where the elliptical shape prior is in an augmented state space and Poincare´ sectioned. Parameters of the ellipse are estimated, where a projection of a center of the ellipse onto an observed respiratory position provides a real-time estimation for baseline drift of the respiratory motion.

### CROSS-REFERENCE TO RELATED APPLICATIONS

This application claims priority from U.S. Provisional Patent Application 61/270,648 filed Jul. 9, 2009, which is incorporated herein by reference.

### FIELD OF THE INVENTION

The invention relates to radiotherapy. In particular, the invention relates to a method of characterizing, estimating and predicting respiratory motion in real-time during a radiotherapy treatment plan.

### BACKGROUND OF THE INVENTION

To precisely ablate a tumor in radiation therapy, it is important to locate the tumor position in real-time during treatment. However, respiration-induced tumor motions are difficult to track. They are semi-periodic and exhibit variations in baseline, frequency and their fundamental pattern that includes oscillatory amplitude and shape. Current radiotherapy techniques enable highly targeted dose delivery. However, internal organ motion, particularly motion caused by respiration, makes it challenging to track or predict the location of the target volume. Multiple studies have shown that target motion results in observable degradation in the treatment effectiveness and accuracy, where treatment margins are often used to account for the effect of target motion. To further reduce treatment margin in order to spare normal tissue radiation, passive or active breath holding have been applied. As an alternative, respiratory gating has been applied to reduce the tumor motion during beam-on time by limiting radiation exposure to only a portion of the breathing cycle. Dynamically tracking and compensating for the target motion have the potential to improve both delivery efficiency and accuracy. For both gating and dynamic compensation, it is crucial to precisely locate the target position in real-time. Observation noise and system delay require estimation schemes that are robust and capable of prediction. Linear prediction models and the many variants therein often have difficulty in incorporating the semi-periodic structure. On the other hand, a purely periodic model does not sufficiently account for variations, where deviation from periodicity is considered ‘abnormal’ or ‘irregular’.

What is needed is a method of tracking respiratory motion in real-time during a radiotherapy treatment plan that accounts for variations in respiratory baseline motion, respiratory frequency and the fundamental respiratory pattern that includes oscillatory amplitude and shape.

### SUMMARY OF THE INVENTION

To address the shortcomings in the art, a method of profiling respiratory motion is provided that includes estimating a temporal respiratory pattern, using a warping function to map the temporal respiratory pattern to a corresponding phase value, using a baseline drift function to determine drift in the temporal respiratory pattern, and noise filtering the temporal respiratory pattern, where variations in a respiratory motion are provided.

In one aspect of the invention, the estimate of the temporal respiratory pattern is obtained by applying an inverse-warping function to respiratory observations and performing a minimum mean-squared error estimation to results of the inverse-warping.

In another aspect of the invention, the warping function includes combining an elliptical shape prior for providing iso-phase events in real-time, where the elliptical shape prior is in an augmented state space and Poincare´ sectioned. In one aspect, parameters of the ellipse are estimated, where a projection of a center of the ellipse onto an observed respiratory position provides a real-time estimation for baseline drift of the respiratory motion.

According to a further aspect of the invention, an observed breathing trajectory includes a periodic fundamental pattern, where the periodic fundamental pattern is frequency modulated by the phase warping function.

In yet another aspect of the invention, an observed breathing trajectory includes a periodic fundamental pattern, where the periodic fundamental pattern is displacement modulated by the baseline drift function.

In one aspect of the invention, variations in a respiratory baseline, a respiratory frequency and a fundamental respiratory pattern are decoupled.

In another aspect of the invention, respiratory baseline, a respiratory frequency and a fundamental respiratory pattern are filtered to achieve denoising, where the denoising includes a smoothness assumption.

In a further aspect of the invention, respiratory baseline, a respiratory frequency and a respiratory fundamental pattern are assembled back into an original signal space to obtain respiratory prediction results.

According to yet another aspect of the invention, variation in the respiratory motion is modeled as the composite result of baseline drift in the respiratory pattern, frequency variation in the respiratory pattern, fundamental change in the respiratory pattern and additive random observation noise in the respiratory pattern.

In one aspect of the invention, phase estimation includes augmentation of respiratory pattern observations, ellipse fitting the augmented respiratory pattern observations, Poincare´ sectioning the augmented respiratory pattern observations, mapping the Poincare´ intersections back to a time-displacement relation as iso-phase points, and providing linear interpolation, and/or linear extrapolation to generate a continuous phase estimation. According to another aspect an elliptical shape is combined prior in an augmented state space and the Poincare´ sectioning to automatically produce the iso-phase points in real-time.

In another aspect of the invention, a minimum mean-squared error (MMSE) is applied to a single period of the temporal respiratory pattern to provide a globally static fundamental pattern, where a discount factor is applied to the globally static fundamental pattern to account for a temporally varying oscillatory breathing magnitude. In one aspect, the discount factor includes a set of (0, 1), where when the discount factor equals 0 the globally static fundamental pattern is a set of most recent respiratory samples, where when the discount factor equals 1 the globally static fundamental pattern is a weighting of all the respiratory samples.

In another aspect of the invention, respiratory motion is characterize by profiling the temporal respiratory pattern to obtain a continuous prediction of a target position over a range of horizons.

### BRIEF DESCRIPTION OF THE DRAWINGS

FIG. 1 shows a schematic drawing of the decomposition process, according to the current invention.

FIG. 2 shows a flow diagram of the online phase estimation procedure according to the current invention.

### DETAILED DESCRIPTION

The current invention provides a method of decomposing variations in respiratory baseline motion, frequency and fundamental pattern (oscillatory amplitude and shape) from discrete observations in real-time. Baseline drift, frequency (equivalently phase) variation and fundamental pattern change characterize different aspects of respiratory motion and have distinctive clinical indications. Furthermore, smoothness is a valid assumption for each one of these components in their own spaces, and facilitates effective extrapolation for the purpose of estimation and prediction. This process hereafter is referred to as ‘profiling’ to reflect the integration of information extraction, decomposition, processing and recovery. The current method has three major elements: (1) real-time baseline and phase estimation based on elliptical shape tracking in augmented state space and Poincare sectioning principle; (2) estimation of the fundamental pattern by unwarping the observation with phase estimate from the previous step; (3) filtering of individual components and assembly in the original temporal displacement signal space. For the purpose of prediction, the results are comparable to what one would expect from a human operator. The method of the current invention is fully unsupervised and data driven, making it ideal for applications requiring economy, efficiency and flexibility.

In one aspect, the observed breathing trajectory is considered as a periodic fundamental pattern that is frequency modulated by a phase warping function and displacement modulated by a baseline drift function. The observation is corrupted by independent additive noise, which is addressed by the current invention. Note that it is reasonable to assume the phase warping functions and the baseline drifts to be piecewise smooth: the smoothness in the phase warping function corresponds to the semi-periodic structure. For tracking and prediction, the following procedure is applied: first, the variations in baseline, frequency and fundamental pattern are decoupled; second, each component is filtered to achieve denoising or prediction, based on a smoothness assumption; finally, the components are assembled back into the original signal space to obtain the ultimate tracking or prediction results.

According to the current invention, the decomposition module produces real-time estimates for baseline drift, frequency variation and fundamental pattern, which are of clinical significance in their own aspects. The baseline drift estimate serves as an indicator for abrupt patient movement, and may be used to trigger interruption of treatment and/or adjustment of patient placement. For treatment with multileaf collimator (MLC), because the physical configuration of the leaves limits the system's tracking ability perpendicular to the leaf track, and it is more reasonable to track the baseline position along such a direction. Large abrupt changes in breathing frequency typically indicate abnormalities and require clinical attention. The fundamental pattern is of its own merit. For lung tumors, radiotherapy planning can be performed based on a pre-treatment 4D CT scan, and it is implicitly assumed that the target motion during the treatment closely resembles that observed in the planning data set. Keeping track of the fundamental pattern provides a way to validate this assumption in real-time: when the patient's breathing pattern deviates significantly from the planning trace, it is necessary to adjust the treatment delivery plan accordingly, or even re-plan in some cases.

According to the invention, the variation in respiratory motion is modeled as the composite result of four sources: baseline drift, frequency variation, fundamental pattern change and additive random observation noise. Each of these factors characterizes a different aspect of respiratory motion and has distinct physiological interpretation and clinical consequences. According to one aspect, an online profiling system is presented that provides real-time estimating of baseline drift, frequency variation and pattern change.

In another aspect of the invention, respiratory motion is characterize by profiling the temporal respiratory pattern to obtain a continuous prediction of a target position over a range of horizons.

A theoretical foundation of the invention is provided. For simplicity, the derivation for the case of scalar observations is provided, but all analyses generalize to high-dimensional situations naturally. The different types of variations in breathing are reflected in the discrete observations according to the generative model shown below, where a fundamental pattern that is modulated both in frequency and in amplitude, and corrupted by additive noise before discrete samples are obtained.

Mathematically, the generative model for the position observation s(t) is given by

sᵢ=[f(φ(t))+b(t)+ω(t)]|ₜ₌ₜᵢ, i=1, 2, . . . ,

where
f: 2π-periodic fundamental pattern.
φ: the phase warping function that maps the time tag to the corresponding phase value.

As a warping function, it is monotonically non-increasing.
b: temporally piecewise slow varying baseline drift function.
ω: independent zero mean additive noise.

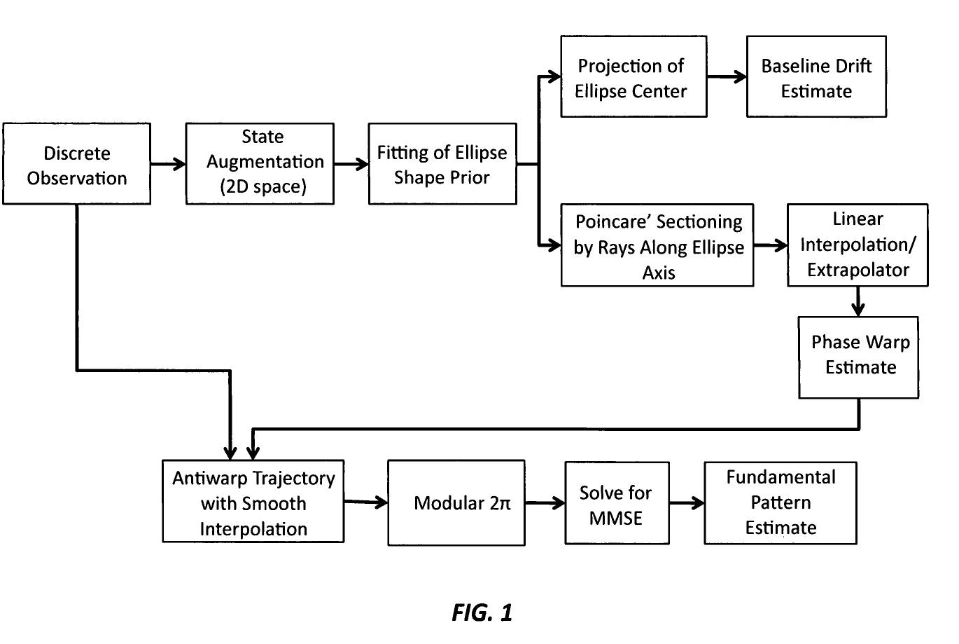

For profiling, the instantaneous breathing pattern is characterized by estimating (f, φ, b) simultaneously. This is an ill-posed problem. A real-time approach to systematically estimate the baseline drift b from the observations is provided. The decomposition of fundamental pattern f and warping map φ will be solved, where given the phase warp φ, an estimate for the fundamental pattern f can be obtained by first ‘inverse-warping’ the observations with φ⁻¹, and then performing minimum mean-squared error (MMSE) estimation on f, based on the periodicity assumption, as shown in the decomposition process of FIG. 1.

A continuous real-time phase estimate is acquired from noisy discrete observations. A linear phase method and its applicability in respiratory motion analysis are summarized hereafter. Given marker events that identify the completion of a cycle (e.g., the R-peaks of an electrocardiogram), the linear phase is defined via interpolation between the neighboring marker events:

$${φ}^{L} ( t ) = 2 π \frac{t - {t}_{n}}{{t}_{n + 1} - {t}_{n}} + 2 π n ( {t}_{n} \leq t < {t}_{n + 1} ) ,$$

where tₙ represents the time tag for the nth marker event. An advantage of the linear interpolating phase is that it is locally defined, where perturbations and errors do not propagate over time. It's purpose is to efficiently identify the marker events when applying the linear phase method to a breathing trajectory. Historically, the peaks and troughs have been identified from a low-pass-filtered breathing trajectory, and subsequently an average tumor trajectory (ATT) is obtained. There exists, however, an intrinsic dilemma with such an approach: the identification of extreme values is very sensitive to noise, due to diminished values of local gradients; while noise suppression via low-pass filtering introduces uncertainty to the detected extreme locations.

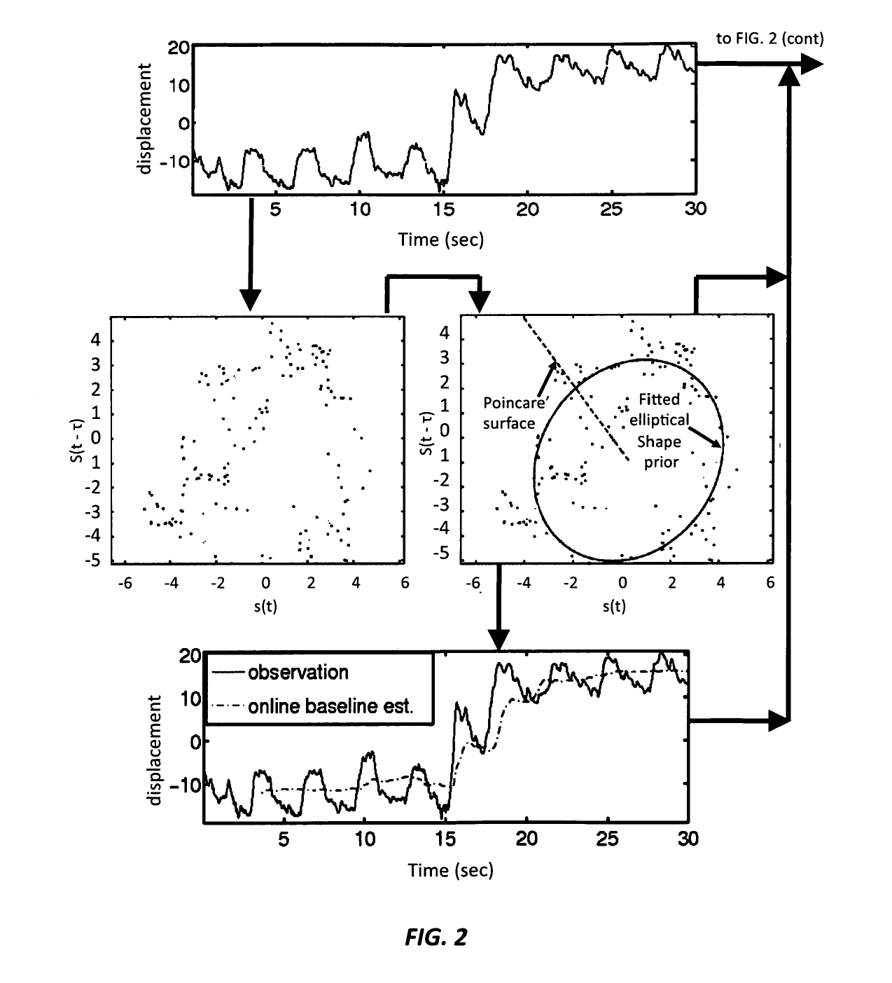

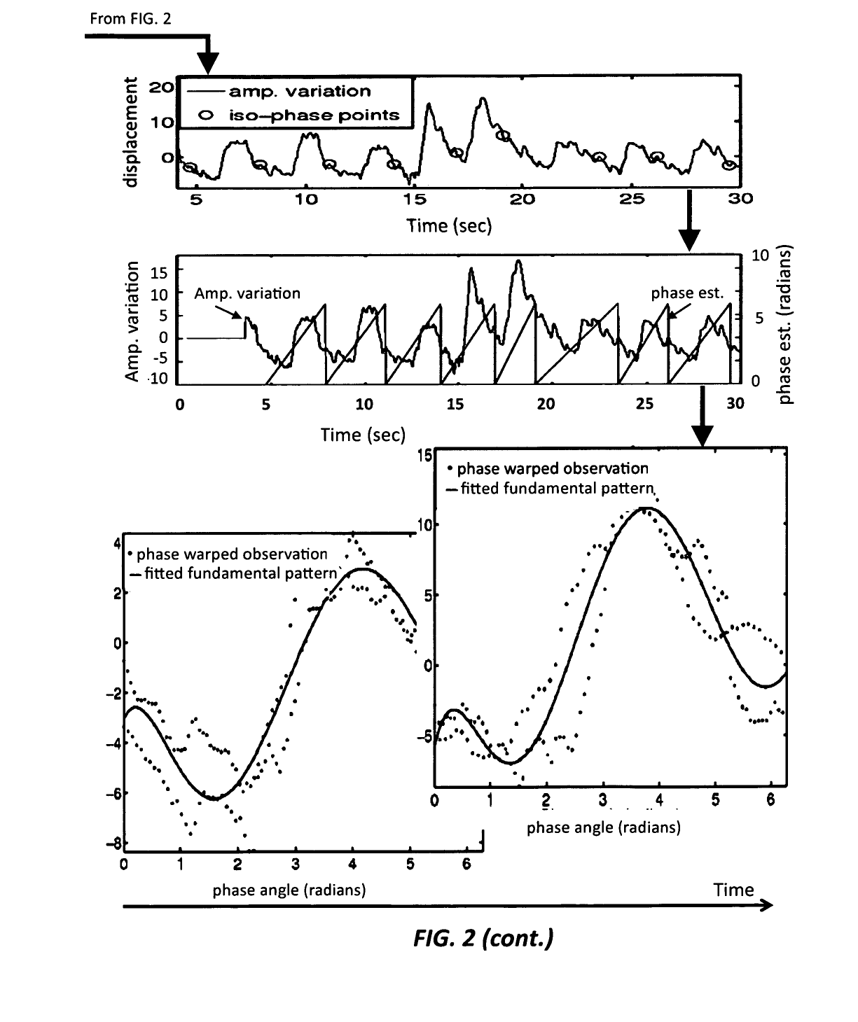

For the purpose of warping, it suffices to identify iso-phase points. According to one aspect of the invention, an elliptical shape is combined prior in an augmented state space and Poincare' sectioning idea to automatically produce the iso-phase events in real-time. FIG. 2 show flow diagrams of the basic procedure for the online phase estimation.

A state vector is formed using the current sᵢ and a past observation of a fixed temporal delay sᵢ₋ₖ to capture the first-order system dynamics. Assuming uniform sampling with interval Δ second, the discrete time lag k corresponds to a delay of τ=k Δ second. This augmentation naturally distinguishes different stages of hysteresis. The elliptical shape prior provides enough information in characterizing the semi-periodicity intrinsic to the time—displacement observations. The procedure for estimating the ellipse parameters is summarized below.

Let (x, y) denote the coordinates of a point in the two-dimensional augmented state space, and define vector z=(x² xy y² x y 1). The point (x, y) falls on the ellipse parameterized by a=(a b c d e f) if and only if it satisfies the following quadratic curve equation:

F(a, z)=a^{T} z=ax²+bxy+cy²+dx+ey+f=0

with negative discriminant, i.e., b²−4ac<0.

The optimal coefficient a is the eigenvector that corresponds to the only positive eigenvalue for the generalized eigendecomposition of the pair (S, C), where

$$S {=}^{Δ} {\sum }_{i} {z}_{i} {z}_{i}^{T} ,$$

where zᵢ is generated with (x=sᵢ, y=sᵢ₋ₖ), and

$$C = [ \begin{matrix}\tilde{C} & {0}_{3 \times 3} \\ {0}_{3 \times 3} & {0}_{3 \times 3}\end{matrix} ] , with \tilde{C} {=}^{Δ} [ \begin{matrix}0 & 0 & 2 \\ 0 & - 1 & 0 \\ 2 & 0 & 0\end{matrix} ] .$$

The conversion from the quadratic parameterization with a to the standard Euclidean representation of the ellipse is accomplished by standard methods. Here, the converted parameters are called (x₀, y₀, r₁, r₂, θ), where (x₀, y₀) is the estimated center of the ellips r₁ and r₂ are the lengths of the major and minor axes respectively, and θ is the angular direction of the major axis. In other words, any point on the ellipse satisfies

$${\{ \frac{cos θ ( x - {x}_{0} ) + sin θ ( y - {y}_{0} )}{{r}_{1}} \}}^{2} + {\{ \frac{- sin θ ( x - {x}_{0} ) + cos θ ( y - {y}_{0} )}{{r}_{2}} \}}^{2} = 1.$$

The projection of the center of the ellipse onto the s(t) axis, i.e., x₀, provides a reasonably good estimate for baseline drift in real-time.

For applying the linear phase estimator it is necessary to identify marker events and pick peaks and troughs of the breathing pattern. It is sufficient to obtain marker events for a definite but arbitrary phase, as discussed hereafter.

To put the Poincare´ sectioning method into context, the Poincare´-Bendixson theorem for planar dynamic systems states: “Given a differentiable real dynamic system defined on an open and simply connected subset of the plane, then every non-empty compact ω-limit set of an orbit, which contains no fixed points, is a periodic orbit.”

Based on this theorem, the trajectory in the augmented state space is a periodic orbit, as there exists no fixed point in the state evolution process. It is important to distinguish between the ‘periodic orbit’ in the state space, referred to as in the above theorem, and the more intuitive ‘semi-periodicity’ with respect to the time evolution. The former only concerns the pattern of the augmented samples in the state space, and is oblivious of the exact time scale. To illustrate this fundamental difference, consider a trajectory of samples according to

$$\begin{matrix}x ( t ) = sin ( 2 π t / {T}_{n} ) & for {\sum }_{i = 1}^{n - 1} {T}_{i} \leq t < {\sum }_{i = 1}^{n} {T}_{i} ; \\ y ( t ) = cos ( 2 π t / {T}_{n} ) & for {\sum }_{i = 1}^{n - 1} {T}_{i} \leq t < {\sum }_{i = 1}^{n} {T}_{i} ,\end{matrix}$$

where {Tᵢ} are positive numbers that control the local speed of evolution. Note that the trajectory defined above is not periodic in time for nonidentical Tᵢ's. On the other hand, this produces a periodic orbit in the state space, which can be expressed as x²+y²=1, and discrete samples from this stipulated generative process fall on this orbit. Therefore, noisy observations are expected from semi-periodic signal to generate scatter plots about an orbit in the state space, oblivious to its own frequency variation. For the purpose of identifying the iso-phase events, the invisibility of pure frequency variation is used, and regarded as an implicit phase warping procedure. Then a Poincare´ section is generated by choosing a Poincare´ plane and recording the intersections of such plane with the given trajectory. If a system has a natural period associated with it, then the Poincare´ plane corresponds to a definite (but arbitrarily) chosen phase. For breathing motion, there is often no definite natural period with respect to time, since the frequency is subject to small variation, yet there exists one in the state space defined above, illustrated by the orbit. The intersecting points between the trajectory and the Poincare´ plane are subsequently identified as iso-phase events.

The placement of the Poincare´ surface is of high relevance for the usefulness of the extracted intersections. An optimal surface maximizes the number of intersections, or equivalently, minimizes the time interval between them. As presented above the trajectory is a periodic orbit according to the Poincare´-Bendixson theorem, so it suffices to find a plane that intersects each period exactly once. An elliptical shape prior is provided, which reasonably approximates the structure of the trajectory in the state space. This leads to a simple construction of the Poincare surface: a ray starting from the center of the estimated ellipse center. To maintain consistency of such a choice, the relative direction of the ray is fixed to the orientation of the ellipse θ in

$${\{ \frac{cos θ ( x - {x}_{0} ) + sin θ ( y - {y}_{0} )}{{r}_{1}} \}}^{2} + {\{ \frac{- sin θ ( x - {x}_{0} ) + cos θ ( y - {y}_{0} )}{{r}_{2}} \}}^{2} = 1 ,$$

which is constantly changing with new observations. Note that the requirement for a legitimate Poincare´ section is much less stringent than that for peaks/trough: it only requires consistency, but not extrema. According to the current invention, it is also feasible to use multiple Poincare´ surfaces if desired.

Once the intersections between the Poincare´ surface and the trajectory are identified as iso-phase events, the phases before the last marker event can be obtained by linear interpolation

$${φ}^{L} ( t ) = 2 π \frac{t - {t}_{n}}{{t}_{n + 1} - {t}_{n}} + 2 π n ( {t}_{n} \leq t < {t}_{n + 1} ) .$$

Linear extrapolation is used to extend phase estimates for time tags that are beyond the last marker event as

$$φ ( t ) = \{ \begin{matrix}2 π \frac{t - {t}_{n}}{{t}_{n + 1} - {t}_{n}} + 2 π n & for {t}_{n} \leq t < {t}_{n + 1} ; \\ 2 π \frac{t - {t}_{N}}{{t}_{N} - {t}_{N - 1}} + 2 π N & for {t}_{N} < t .\end{matrix}$$

Once the baseline drift b and the phase φ, the estimation of fundamental pattern f, based on minimum mean-squared error (MMSE) estimation, is provided with the following steps:
Obtain a continuous time—displacement function from discrete samples {sᵢ}. With finite noisy observations sᵢ, the continuous trajectory \tilde{s} is modeled with linear combinations of synthesis functions:

$$\tilde{s} ( t ) = {\sum }_{k} {c}_{k} η ( t - k Δ ) .$$

With finite noisy number of observations sᵢ, it is advantageous to use a non-interpolant synthesis function η, rather than the infinitely supported sinc or cardinal splines. In this implementation, the third-order B-spline is adopted for the purpose of interpolation using recursive filtering.
Estimate the fundamental pattern f by inverse warping. Because f is a 2π periodic function, an equivalent representation is provided by restricting it to a single period: \tilde{f}=f|_{[0,2π)}, and the MMSE estimate is the average:

\tilde{f}(θ)=mean{\tilde{s}(t): t=φ⁻¹(2kπ+θ) for some k ε [symbol] }.

The above estimation is for a globally static fundamental pattern. In some cases, the oscillatory magnitude of the breathing is also temporally varying, and, according to the invention, it may be desirable to apply adaptive learning. Mathematically, a discount factor λ ε (0, 1) is introduced, and the goal is to find the \tilde{f} such that

$${\tilde{f}}^{*} ( θ ) = arg {min}_{\overline{f} ( θ )} {\int }_{θ = 0}^{2 π} {\sum }_{i = 1}^{K ( θ )} {λ}^{K ( θ ) - i} {‖ s ( {φ}^{- 1} ( 2 π i + θ ) ) - \overline{f} ( θ ) ‖}^{2} ,$$

where K(θ) is the biggest integer such that K(θ)2π+θ≦φ(t). The adaptive weight λ controls the discounting ratio between past samples and the more current ones. It reflects the tradeoff between system responsiveness and noise sensitivity, and thus is also called a ‘forgetting factor’. In particular, λ=0 indicates a myopic estimation using only the most recent samples, and λ=1 corresponds to weighting all the samples equally, ignoring their relative temporal distance to the current instant.

The solution to \tilde{f}*(θ) above is given by the weighted average of historical data corresponding to the same phase:

$${\tilde{f}}_{t}^{*} ( θ ) = \frac{1}{{C}_{t} ( θ )} {\sum }_{i = 1}^{K ( θ )} {λ}^{K ( θ ) - i} s ( {φ}^{- 1} ( 2 π i + θ ) ) ,$$

where

$${C}_{t} ( θ ) = {\sum }_{i = 1}^{K ( θ )} {λ}^{K ( θ ) - i}$$

is the normalization factor. Note that the λ=1 case corresponds to real-time averaging, which at the end of the horizon, reduces to

\tilde{f}(θ)=mean{\tilde{s}(t): t=φ⁻¹(2kπ+θ) for some k ε [symbol] }.

The present invention has now been described in accordance with several exemplary embodiments, which are intended to be illustrative in all aspects, rather than restrictive. Thus, the present invention is capable of many variations in detailed implementation, which may be derived from the description contained herein by a person of ordinary skill in the art. For example the piecewise linear form for the phase warping function can be generalized to other functional forms that maintain monotonicity of the warping.

All such variations are considered to be within the scope and spirit of the present invention as defined by the following claims and their legal equivalents.

1. A method of profiling respiratory motion, comprising:
a. estimating a temporal respiratory pattern;
b. using a warping function to map said temporal respiratory pattern to a corresponding phase value;
c. using a baseline drift function to determine drift in said temporal respiratory pattern; and
d. noise filtering said temporal respiratory pattern, wherein variations in a respiratory motion are provided.

2. The method of claim 1, wherein said estimate of said temporal respiratory pattern is obtained by applying an inverse-warping function to respiratory observations and performing a minimum mean-squared error (MMSE) estimation to results of said inverse-warping.

3. The method of claim 1, wherein said warping function comprises combining an elliptical shape prior for providing iso-phase events in real-time, wherein said elliptical shape prior is in an augmented state space and Poincare' sectioned.

4. The method of claim 3, wherein parameters of said ellipse are estimated, wherein a projection of a center of said ellipse onto an observed respiratory position provides a real-time estimation for baseline drift of said respiratory motion.

5. The method of claim 1, wherein an observed breathing trajectory comprises a periodic fundamental pattern, wherein said periodic fundamental pattern is frequency modulated by said phase warping function.

6. The method of claim 1, wherein an observed breathing trajectory comprises a periodic fundamental pattern, wherein said periodic fundamental pattern is displacement modulated by said baseline drift function.

7. The method of claim 1, wherein variations in a respiratory baseline, a respiratory frequency and a fundamental respiratory pattern are decoupled.

8. The method of claim 1, wherein respiratory baseline, a respiratory frequency and a fundamental respiratory pattern are filtered to achieve denoising, wherein said denoising comprises a smoothness assumption.

9. The method of claim 1, wherein respiratory baseline, a respiratory frequency and a respiratory fundamental pattern are assembled back into an original signal space to obtain respiratory prediction results.

10. The method of claim 1, wherein variation in said respiratory motion is modeled as the composite result of baseline drift in said respiratory pattern, frequency variation in said respiratory pattern, fundamental change in said respiratory pattern and additive random observation noise in said respiratory pattern.

11. The method of claim 1, wherein phase estimation comprises augmentation of respiratory pattern observations, ellipse fitting said augmented respiratory pattern observations, Poincare' sectioning said augmented respiratory pattern observations, mapping said Poincare' intersections back to a time-displacement relation as iso-phase points, and i) providing linear interpolation, ii) linear extrapolation or i) and ii) to generate a continuous phase estimation.

12. The method of claim 11, wherein an elliptical shape is combined prior in an augmented state space and said Poincare' sectioning to automatically produce said iso-phase points in real time.

13. The method of claim 1, wherein a minimum mean-squared error (MMSE) is applied to a single period of said temporal respiratory pattern to provide a globally static fundamental pattern, wherein a discount factor is applied to said globally static fundamental pattern to account for a temporally varying oscillatory breathing magnitude.

14. The method of claim 13, wherein said discount factor comprises a set of (0, 1), wherein when said discount factor equals 0 said globally static fundamental pattern comprises a set of most recent respiratory samples, wherein when said discount factor equals 1 said globally static fundamental pattern comprises a weighting of all said respiratory samples.

15. The method of claim 1, wherein said respiratory motion is characterized by profiling said temporal respiratory pattern to obtain a continuous prediction of a target position over a range of horizons.
